(19) United States
(12) Patent Application Publication
(10) Pub. No.: US 2008/0260697 A1
(43) Pub. Date: Oct. 23, 2008

(54) Bacterial Management in Animal Holding Systems

(75) Inventors: Kishore Murthy, Ottawa (CA); Rainer Englehardt, Ottawa (CA)

Correspondence Address: BRINKS, HOFER, GILSON & LIONE, 2801 SLATER ROAD, SUITE 120, MORRISVILLE, NC, 27560, US

(73) Assignee: GANGAGEN LIFE SCIENCES INC., Ottawa, ON (CA)

(21) Appl. No.: 11/915,272

(22) PCT Filed: May 26, 2006
(86) PCT No.: PCT/CA2006/000864

§ 371 Date: May 14, 2008

Related U.S. Application Data
(60) Provisional application No. 60/685,108, filed on May 26, 2005.

(51) Int. Cl. A61K 35/76; A61K 9/00; A61P 31/04

(52) U.S. Cl. 424/93.6; 424/484

(57) ABSTRACT

The present invention is directed to method for reducing a population of a target pathogen in an animal or within a feedlot. The method involves administering one or more than one controlled release bacteriophage strain or phage component, or both, to the animal, so that the one, or more than one bacteriophage strain is released in vivo and adsorbs to the one or more than one target pathogen, thereby reducing the one, or more than one pathogen from the animal. The controlled release bacteriophage strain or phage component may be administered as a treatment dose prior to further processing of the animal, a treatment dose followed by a maintenance dose, or a maintenance dose, to manage feedlot target pathogens.

The present invention relates to methods for reducing bacteria within animal holding systems. More specifically, the present invention provides methods to control pathogenic bacteria within an animal, animal production systems such as a feedlot, rearing enclosure, and the like, or a combination thereof.

### BACKGROUND OF THE INVENTION

Contamination of meat and meat products destined for human consumption is an ongoing problem in the food industry. Of particular concern are Escherichia coli, Salmonella spp., and Campylobacter spp. pathogens, all of which can cause food-borne illnesses in humans. Human illness due to these pathogens is also often caused by the consumption of contaminated meat products including chicken, turkey, beef and pork meats. Such pathogens may also pose a health hazard to humans directly engaged in the production of animals. Also, other pathogens carried in the gastrointestinal tract of animals may a health hazard to the animals directly or indirectly.

The sources of bacterial contamination for pigs, poultry, and cattle are numerous, including unclean bedding and feed. Dry feed used for swine, poultry, cattle and other production animals can be contaminated with bacteria including Salmonella and E. coli. Such contamination can occur during processing, storage or transportation. By example, a common source of contamination of meat and meat products from beef cattle is the abattoir, where contamination of the carcass by bacteria on or in hide, hair or faeces can occur.

In aiming to reduce human food-borne illnesses, research has shifted to pre-slaughter intervention in the live animal. Probiotic, dietary, and anti-pathogen methods have all been used in trying to achieve this goal.

For example, U.S. Pat. No. 5,965,128 (Doyle et al; see also Zhao et al (1998) J Clin Microbiol, 36, 641-647) teaches the use of probiotic bacteria to reduce or prevent the carriage of E. coli O157:H7 in experimentally inoculated cattle. However, the method taught by U.S. Pat. No. 5,965,128 is highly invasive and involves inoculation of cattle via rumen cannulation. Such a method does not provide a convenient method of administration of the probiotic bacteria. Other studies (Brashears et al (2003) J Food Prot, 66, 748-754; Younts-Dahl et al (2004) J Food Prot, 67, 889-893) have shown that supplementation with Lactobacillus, Propionibacterium microbials, or both, can decrease E. coli O157:H7 shedding in cattle, but will not eliminate the pathogen. Similar results are disclosed by Garner and Ware (US 2003/0175305, US 2003/0175306, US 2003/0175307, and WO 2004/030624).

Changes in dietary regimen have also been investigated for reducing pathogen shedding in cattle. Allen et al (U.S. Pat. No. 6,270,812) teaches the use of a seaweed supplement in the feedlot finishing diet to reduce pathogenic E. coli in post-slaughter fecal samples. Others (Braeden et al (2004) J Food Prot, 67, 1824-1828) found that supplementation of the feed with Tasco-14 reduced the prevalence of E. coli O157:H7 in cattle. Still others (Diez-Gonzalez et al (1998) Science, 281, 1666-1668) have shown that abrupt switching of grain-fed cattle to a hay diet reduces the E. coli population. However, the magnitude of dietary effects on levels of E. coli O157:H7 in cattle varies, and the results remain controversial.

Specific targeting and elimination of pathogens from cattle, poultry and swine is another approach in the battle against food-borne illness. For example, antibiotics such as neomycin significantly reduced fecal shedding of E. coli O157:H7 in cattle (Ransom et al (2003) Research Fact Sheet, National Cattleman's Beef Association, Centennial Colo.). Even though neomycin is not used in human medicine, the extensive use of this antibiotic may result in the development of resistance to related antibiotics such as gentamycin, kanamycin etc. commonly used in human medicine. A European ban on the prophylactic use of antibiotics in food and the possibility of widespread dissemination of antibiotic resistance discourages the use of such compounds.

Bacteriophages have also been considered for use in treatment of animal wastes. Bacteriophages (or “phages”) are bacterial viruses that specifically infect and kill bacteria, and are widely distributed in nature. Phages recognize receptors on the bacterial surface, attach to them and inject their genetic material into the host cell. They degrade the host bacteria's DNA and synthesise their own genetic material and required coat proteins, then re-assemble multiple copies of bacteriophage particles before bursting the cell. The released bacteriophages will then infect and destroy additional bacteria in the surrounding environment. This process continues until all the bacteria are eliminated from the system.

U.S. Pat. No. 6,656,463 discloses reduction of Salmonella populations within swine using Felix 0-1 phage. Smith et al (J Gen Microbiol (1987) 133, 1111-1126) have shown that phages may be useful in controlling enterotoxigenic E. coli infections in livestock. The study showed that strain-specific phages could cure or prevent E. coli diarrhea in calves by a single oral dose, or by spraying of the litter with phages. However, the phages were only efficient if administered prior to or simultaneously with administration of E. coli. Furthermore, the pathogen used by Smith et al is distinct from E. coli O157:H7, and the phages found to be effective in this study will not recognize E. coli O157:H7.

Kudva et al (Appi Env Microbiol (1999) 65, 3767-3773) showed that phages were efficient in reducing the amount of or clearing E. coli O157:H7 from cultures. Specifically, no single phage could clear an E. coli O157:H7 culture, however a mixture of three 0157-specific phages was capable of eliminating the bacteria from cultures. However, the in vitro experiments of Kudva et al (1999) do not indicate that such phages would be efficient in controlling E. coli O157:H7 in vivo in livestock. Other in vitro studies have been reported; however, no or low, effectiveness is observed when these phage are tested in vivo (see Callaway T. R. et al. (2004) J Animal Sci. 82(E.Suppl):E93-99 for review). Further improvements need to be made to effectively use phages to reduce E. coli 0157 infection of cattle.

U.S. Pat. No. 6,485,902 (Waddell et al) teaches the use of specific bacteriophages to reduce the levels of E. coli O157:H7 in the gastrointestinal tract of cattle. A mixture of six phages was administered orally in high dosages to calves prior to and after challenge with E. coli O157:H7. This study showed that the shedding of E. coli O157:H7 in feces was reduced compared to calves not receiving phages. However, the high dosages required by the method suggest inactivation of the bacteriophages in the gastrointestinal tract.

Despite improved post-slaughter sanitation, food-borne illnesses in humans due to contaminated meat and meat products is an ongoing problem in the food industry. Generally, these illnesses can be attributed to E. coli, Salmonella, and/or Campylobacter. Various methods have been investigated for reducing the incidence of pathogens in poultry, swine and cattle, including: supplementation with probiotic bacteria, dietary supplements, dietary changes, antibiotics, and bacteriophages. However, most methods are economically non-viable, controversial, or unproven. Furthermore, most studies use experimentally infected animals, which may not truly reflect the effect of anti-pathogen treatment on naturally infected animals.

### SUMMARY OF THE INVENTION

The present invention relates to a method for reducing pathogenic bacteria within animal holding systems.

It is an object of the present invention to provide a method for reducing bacteria within an animal holding system.

The present invention provides a method (A) for reducing a population of one, or more than one target pathogen present in an animal comprising, administering one or more than one controlled release bacteriophage strain, phage components, or a combination thereof, to the animal, such that the one or more than one controlled release bacteriophage strain, phage components, or combination thereof, is released in vivo, and adsorbs onto and reduces the population of the one or more than one target pathogen from the animal.

In the process as described above, the one or more than one controlled release bacteriophage, phage component or combination thereof, may be administered in a treatment dosage of about 10⁷ to about 10¹³ pfu per animal per day from about 1 to about 12 days. Alternatively, the one, or more than one controlled release bacteriophage strain, or phage components, may be administered in a maintenance dosage of about 10⁵ to about 10¹⁰ pfu per animal per day for the next 30 to 90 days. In yet another alternative, the one or more than one controlled release bacteriophage strain, or phage components, may initially be administered in a treatment dosage of about 10⁷ to about 10¹³ pfu per animal per day from about 1 to about 12 days, followed by a maintenance dosage of about 10⁵ to about 10¹⁰ pfu per animal per day for the next 30 to 90 days.

The controlled release bacteriophage or phage components described above may be administered by adding to animal feed or drinking water, by inhalation, or injection either intramuscular, intraperitoneal, or intrathecal, or by administering rectally, topically, or a combination of these methods.

The present invention also provides a method (B) for reducing a population of one, or more than one target pathogen present within a holding system, comprising, administering one or more than one controlled release bacteriophage strain, or phage components to animal feed, drinking water, an animal, or a combination thereof, such that the one or more than one controlled release bacteriophage strain, or phage components, is released within the feed, the drinking water, a digestive tract of the animal, manure, or a combination thereof, and adsorbs to, and kills, the target pathogen in the surrounding environment, thereby reducing the population of the one or more than one target pathogen within the holding system. The controlled release bacteriophage or phage components described above may be administered by adding to animal feed or drinking water, by inhalation, or injection either intramuscular, intraperitoneal, or intrathecal, or by administering rectally, topically, or a combination of these methods. The holding system may include, but is not limited to a feedlot, a holding pen prior to slaughter, a rearing enclosure, including for example a rearing barn or rearing pen, a petting zoo, open or closed aquaculture systems, other animal housing quarters, and the like.

Phages can be administered to the animals when they are brought into a holding system, for example a holding or rearing enclosure, such as a feedlot, from different farms with varied pathogen control status. Administering phages to the animals at a dose of 10⁷ to about 10¹³ pfu per animal per day for 1 to about 12 days. This could be followed by a maintenance dose of 105 to about 10¹⁰ pfu per animal per day for the next 30 to 90 days. Using this protocol helps reduce the overall contamination of the farm by this pathogen.

The present invention also provides a method for preventing the spread of infections in an animal caused by one or more than target pathogen. The method comprises administering one or more than one bacteriophage strain, phage component, or both, to the animal, such that the one, or more than one bacteriophage strain, phage component, or both, is released within the digestive tract of the animal, attach to and kill the target pathogen, thereby reducing the population of the one or more than one target pathogen within animal waste. The target pathogen may be E. coli O157:H7, Staphylococcus aureus, Treponema, or another pathogen carried in the gastrointestinal tract, or a combination thereof. The one or more than one bacteriophage strain, phage component, or both, may be provided as a controlled release bacteriophage strain, phage component, or both.

The present invention further provides treatment protocols for the reduction of pathogens, for example but not limited to E. coli, Salmonella, Campylobacter, and Staphylococcus, in animals. Animals to be shipped, or going to slaughter, may be treated with one or more than one controlled release bacteriophage strain, phage component, or both, 5-7 days before being shipped. Using this approach, the pathogen level of the animals will be reduced to low levels by day 3-5 of the treatment, thus allowing safe shipment of the animals from approximately day 4 of treatment onwards. This provides a “safe shipping and processing” window during which the animals from the holding system, for example but not limited to a holding or rearing enclosure, such as a feedlot, can be shipped and processed safely.

In addition, the present invention provides a use of one or more than one controlled release bacteriophage strain, phage component, or both, for delivery to animal waste to prevent the spread of bacterial infections through the waste. The one or more than one controlled release bacteriophage strain, phage component, or both, may be delivered directly to the waste (ex vivo) in an un-encapsulated form, or may be administered to the animal in a controlled release form for delivery to the waste through the animal's gut.

The use of bacteriophages for reducing pathogenic bacteria in animals, or within animals, and animal holding systems including petting zoos, and holding or rearing enclosure such as a feedlot, will help increase the safety of food sources as well as help reduce pathogen contamination of agricultural produce, source water, pets, and the environment in general. For example, in addition to reducing the spread of E. coli O157:H7, which can cause serious health issues in humans, bacteriophages can also reduce the counts of Staphylococcus aureus, which can infect the teats and udder of cattle and cause mastitis. Furthermore, Treponema infections causing hoof disease may be treated in this manner by acting as a foot bath when the animals are walking in the pen. Target pathogen specific bacteriophage, phage components or both, can be safely administered to animals without affecting the non-pathogenic bacterial flora naturally present in the animal or the environment.

This process overcomes the disadvantages of the prior art by treating animals with safe bacteriophages in an effective delivery system. Furthermore, by administering controlled release bacteriophage or phage components, residence time of the bacteriophage or phage components in vivo can be adjusted to ensure a viable and sustained level of the bacteriophage population or phage component within the digestive tract and gut of the animal for a desired period of time, and if desired within animal waste to manage animal, animal holding systems including holding or rearing enclosure, such as a feedlot, or both, to target pathogen populations. The bacteriophage or components of the bacteriophage lack toxins or other potentially environmentally harmful compounds. These highly efficacious bacteriophages provide an economically viable and safe means for controlling bacterial populations within livestock, pets, and other animals as well as the animal holding systems including holding or rearing enclosures, such as a feedlots, and other animal housing quarters (especially in high-intensity farming) and the surrounding environments.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows the titer of phage applied to the skim milk powder (Before) and that obtained after immobilization and resuspension (After).

FIG. 2 shows the titer of phage applied to the soya protein powder (Before) and that obtained after immobilization and resuspension (After).

FIG. 3 shows the effect of encapsulation on bacteriophage activity. Phage titers before and after encapsulation are shown.

FIG. 4A shows the effect of low pH on the stability of encapsulated phages. Encapsulated phage titers were determined before and after grinding. All phage concentrations have been corrected for the weight of encapsulated material. FIG. 4B shows the effect of low pH on the infectivity of phage. The phages were neither immobilized nor encapsulated.

FIG. 5 shows stability of encapsulated immobilized phages over a period of 4.5 months (131 days) and 10 months (311 days) when stored at room temperature (RT) or at 4° C., respectively.

FIG. 6 shows the reduction of E. Coli O157:H7 shedding in phage-treated animals compared to control animals, over a study period of 10 days.

FIG. 7 shows the reduction in number of E. coli O157:H7 positive animals in the phage-treated group compared to control group, over a 10-day period.

FIG. 8 shows the level of free bacteriophage shed in the manure of treated animals over a period of 10 days.

FIG. 9 shows a representative RFLP pattern of the administered phages before and after passing through the animal (cattle). The patterns were obtained using three different enzymes.

FIG. 10 shows suggested protocol for the treatment of cattle prior to shipment for slaughter. Suggested safe shipping and processing periods are also identified.

### DESCRIPTION OF PREFERRED EMBODIMENT

The present invention relates to a method for reducing pathogenic bacteria within animal holding systems. More specifically, the present invention provides methods to control pathogenic bacteria within an animal, animal production systems such as a feedlot, rearing enclosure, and the like, or a combination thereof.

The following description is of a preferred embodiment. The present invention provides a method for reducing a population of one, or more than one target pathogen present in an animal, comprising, administering one or more than one controlled release bacteriophage strain, or phage components, to the animal, such that the one, or more than one controlled release bacteriophage strain, or phage components, is released in vivo and within a gut, and acts to clear the one or more than one pathogen from the animal.

Also provided is a method for reducing a population of one, or more than one target pathogen present within an animal holding system, for example but not limited to a holding or rearing enclosure, such as a feedlot, comprising, providing one or more than one controlled release bacteriophage strain and/or phage components to animal feed, drinking water, an animal, or a combination thereof, such that the one or more than one controlled release bacteriophage strain, phage component, or both, is released within the feed, the drinking water, a digestive tract of the animal, manure, or a combination thereof, and adsorbs to the target pathogen thereby killing or reducing the population of the one or more than one target pathogen within the animal holding system. Animal holding systems may include, but are not limited to a feedlot, a holding pen or enclosure prior to slaughter, a rearing enclosure, including for example a rearing barn or rearing pen, a petting zoo, open and closed aquaculture systems, other animal housing quarters, and the like.

By the term “animal” or “animals”, it is meant any animal that may be affected by, or carry, a pathogen. For example, but without wishing to be limiting in any manner, animals may include animals for agricultural use; non-limiting examples include, poultry, such as chicken or turkey, and the like; swine; livestock, which term includes all hoofed animals such a horses, goats, sheep, and cattle—including but not limited to beef cattle, dairy cattle and bison—and the like. Animals may also include domesticated animals, for example but not limited to household pets such as cats, dogs, and the like. Another non-limiting example of animals includes various species in aquaculture, such as fish and shellfish.

The term “bacteriophages” or “phages” is well known in the art and generally indicates a virus that infects bacteria. Phages are parasites that multiply inside bacterial cells by using some or all of the host's biosynthetic machinery, and can either be lytic or lysogenic. The bacteriophages used in accordance with the present invention may be any bacteriophage, lytic or lysogenic that is effective against a target pathogen of interest. However, the bacteriophages for use in the present invention are preferably selected to be non-lysogenic, which means that the phage DNA is not incorporated into the host's genomic DNA following phage infection. Phage specific for one or more than one target pathogen may be isolated using standard techniques in the art for example as taught in Maniatis et al (1982, Molecular cloning: a laboratory manual, Cold Spring Harbor Laboratory, Cold Spring Harbor, N.Y.; which is incorporated herein by reference). If desired, a cocktail of different bacteriophage may be used to target one or more than one pathogen as described herein.

Similarly, “phage component” or “phage components” may comprise any phage component including but not limited to the tail, a phage protein, or other constituent molecule or molecular construct that is effective in killing, reducing growth, or reproduction of a target bacteria, or a plurality of target bacteria.

If desired, a cocktail of bacteriophages strains, phage components, or both (also referred to as “bacteriophages and/or phage components), may be used against a single bacterial target, or multiple bacterial targets. By the term “target pathogen” or “target bacteria”, it is meant pathogenic bacteria that may cause illness in humans, animals, fish, birds, or plants. The target bacteria may be any type of bacteria, for example but not limited to the bacterial species and strains of Escherichia coli, Streptococci, Humicola, Salmonella, Campylobacter, Listeria, Lawsonia, Staphylococcus, Pasteurella, Mycobacterium, Hemophilus, Helicobacter, Mycobacterium, Mycoplasmi, Nesseria, Klebsiella, Enterobacter, Proteus, Bactercides, Pseudomas, Borrelius, Citrobacter, Propionobacter, Treponema, Shigella, Enterococcus, Leptospirex, Bacillii including Bacillus anthracis and other bacteria pathogenic to humans or animals. Of interest are bacteria that are known to contaminate animal feeds, liquid animal feeds, or animal holding systems, including, but not limited to holding or rearing enclosures, such as a feedlots, generally. Of particular interest are bacteria that also infect livestock, including swine, and poultry destined for human consumption for example but not limited to Salmonella, Campylobacter and E. coli O157:H7, or any combination thereof. In another non-limiting example, the target pathogen may be E. coli, Staphylococcus, Treponema, or any combination thereof.

The bacteriophages and/or phage components, may be provided in an aqueous solution. The aqueous solution may be any solution suitable for the purpose of the present invention. For example, the bacteriophages and/or phage components, may be provided in water or in an appropriate medium as known in the art, for example LB broth, SM, TM, PBS, TBS or other common buffers. For example, but without wishing to be limiting, the bacteriophages may be stored in LB broth.

The bacteriophages and/or phage components also may be provided in a dry form for admixing with either a liquid animal feed or an animal feed. Examples of dry forms of bacteriophages and/or phage components include but are not limited to lyophilized bacteriophages and/or phage components, bacteriophages and/or phage components that are immobilized on a matrix, bacteriophages and/or phage components that are encapsulated as described below, bacteriophages and/or phage components that are provided in capsule form as described below, bacteriophages and/or phage components that are provided in tablet form as described below, or a combination thereof.

By “controlled release” it is meant that the agent administered to the animal, for example one or more than one bacteriophage and/or phage component is present in a composition comprising various formulations of the one or more than one bacteriophage and/or phage component. For example, the one or more than one bacteriophage and/or phage component may be present in a liquid or dry form comprising, one or more than one bacteriophage and/or phage component, lyophilized bacteriophages or phage components, bacteriophages and/or phage components that are immobilized on a matrix, bacteriophages and/or phage components that are encapsulated, bacteriophages and/or phage components that are provided in capsule form, bacteriophages and/or phage components that are provided in tablet form, bacteriophages and/or phage components that are encapsulated, in capsules, in tablets, or a combination thereof, wherein the encapsulated, capsule, or tablet forms of the bacteriophages and/or phage components comprise compositions that release the bacteriophages and/or phage components at different rates with various regions of a digestive tract of an animal, or within the animal waste. The compositions of the encapsulated, capsule, or tablets may include polymers, waxes, gels, compounds that imbibe water, repel water, or both, fatty acids, sugars, proteins or synthetic materials, to effect release of an agent within the composition in a controlled manner. Various controlled release compositions comprising bacteriophages or phage components may be used so that the bacteriophages and/or phage components may be released prior to administration to an animal, during passage through the digestive tract of the animal, or after leaving the animal.

The immobilized bacteriophages composition of the present invention exhibits desirable storage properties and may be mixed with the feed of animals including, but not limited to livestock, birds, poultry, domestic animals, fish, and shellfish to aid in reducing the shedding of target bacteria. Controlled release bacteriophages and/or phage components, present as a liquid, immobilized, encapsulated, capsulated, tablet or a combination thereof, may be mixed with other additives or supplements applied to animal feed, as part of the daily feed regime, as needed or incorporated into pelleted feed. Thus, settling of the bacteriophages and/or phage components, in the feed could be avoided. Alternatively, the adhesion of the feed or the encapsulated phage and/or both, may be enhanced to provide improved mixing and delivery. The controlled release bacteriophage and/or phage components may also be admixed with drinking water. Additionally, alternate forms of administration, for example but not limited to inhalation, injection, intramuscular, intraperitoneal, intrathecal, vaginal, rectal, topical or a combination thereof, may be used to administer the controlled release bacteriophages, phage components, or both, of the present invention.

Lyophilization of bacteriophage and/or phage components can be carried out using any known lyophilization procedure, for example but not limited to methods disclosed in Clark and Geary (1973, Preservation of bacteriophages by freezing and freeze-drying, Cryobiology, 10, 351-360; Ackermann et al. 2004, Long term bacteriophage preservation, World Federation Culture Collections Newsletter, 38, 35 (which are both incorporated herein by reference).

The bacteriophages, or phages components, or both, may also be provided immobilized onto a matrix. By the term “matrix”, it is meant any suitable solid matrix that is either soluble in water, ingestible or able to be imbibed by an animal, or suitable for use with liquid animal feed. Additionally, the matrix may be non-water-soluble, provided that any absorbed phages can be released from the matrix within an aqueous environment. The matrix should be capable of adsorbing the bacteriophages and/or phage components, onto its surface and releasing the bacteriophages and/or phage components, in an appropriate environment. The bacteriophages and/or phage components, should not adhere so strongly to the matrix that they cannot be released upon appropriate re-suspension in a medium. Preferably, the adsorbed, immobilized bacteriophages and/or phage components, are non-covalently associated with the matrix so that they may be released from the matrix when desired. Non-limiting examples of a matrix that may be used according to the present invention include skim milk powder, soya protein powder, albumin powder, single cell proteins, trehalose, mannitol or other powdered sugar or sugar alcohol, charcoal, latex beads, synthetic plant-derived plastic such as, but not limited to soya plastic or corn plastic, or other inert surfaces, water-soluble carbohydrate-based materials, or a combination thereof. Preferably, the matrix is generally regarded as safe (GRAS).

The bacteriophages, or phage components, or both, in aqueous solution may be applied to the matrix by any method known in the art, for example dripping or spraying, provided that the amount of the matrix exceeds the amount of aqueous bacteriophage and/or phage components, solution. It is preferred that the matrix remain in a dry or semi-dry state, and that a liquid suspension of bacteriophages (and/or phage components) and matrix is not formed. Once the bacteriophage solution is added to the matrix, the matrix may be mixed using a mechanical device and allowing it to air-dry. Bacteriophage may also be immobilized on the solid matrix using commercially available fluid bed granulators and dryers. Of these methods, spraying the bacteriophage solution over the matrix is preferred.

The antibacterial composition comprising immobilized bacteriophages, or phage components, or both, and matrix may be dried at a temperature from about 0° C. to about 50° C. or any amount therebetween, for example at a temperature of 0, 2, 4, 6, 8, 10, 12, 14, 16, 18, 20, 22, 24, 26, 28, 30, 32, 34, 36, 38, 40, 42, 44, 46, 48, or 50° C. or any amount therebetween. For example, the antibacterial composition may be dried at a temperature from about 10° C. to about 30° C., or any amount therebetween, or from about 15° C. to about 25° C. or any amount therebetween. The drying process may also be accelerated by providing a flow of air over or through the antibacterial composition. Alternatively, drying may be performed by heating the immobilized material under vacuum.

After a period of drying, additional aqueous solution may be applied to the matrix if desired, and the matrix re-dried. This process may be repeated as required to obtain the desired amount of phage on the matrix. The titer of phage on the matrix can be readily determined using standard techniques.

The immobilized or lyophilized bacteriophages and/or phage components, may also be encapsulated prior to administration to an animal as a feed additive. By “encapsulated”, it is meant that the immobilized phages, or phage components, or both, are coated with a substance that increases the phages' resistance to the physico-chemical stresses of its environment. The immobilized phages and/or phage components, may be coated with any substance known in the art, by any suitable method known in the art, for example, but not limited to that disclosed in US publication 2003/0109025 (Durand et al., which is incorporated herein by reference). In this method, micro-drops of the coating substance are injected into a chamber containing a containing one, or more than one immobilized bacteriophage strain, or phage components, or both, of the present invention and rapidly cooled. Alternatively, a coating composition may be admixed with the one, or more than one immobilized bacteriophage and/or phage components, of the present invention, with constant stirring or agitation, and cooled or dried as required.

The coating substance may be any suitable coating substance known in the art. For example, but without wishing to be limiting, the coating substance may comprise a substance with a melting temperature between about 20° C. and about 100° C., for example between about 30° C. and about 80° C., or any temperature therebetween; for example, the melting temperature may be 20, 22, 24, 26, 28, 30, 32, 34, 36, 38, 40, 42, 44, 46, 48, 50, 52, 54, 56, 58, 60, 62, 64, 66, 68, 70 72, 74, 76, 78, 80, 82, 84, 86, 88, 90, 92, 94, 96, 98, or 100° C., or any temperature therebetween. If the coating substance is to be ingested or used for an oral application, then it is preferred that the substance is a food grade substance. Non-limiting examples of such substances include vegetable fatty acids, fatty acids such as palmitic acid and stearic acid, for example Stéarine™, animal waxes, vegetable waxes, for example Carnauba wax and wax derivatives. Other additive molecules may be added to the coating substance; such additive may include antioxidants, sugars, proteins or other synthetic material.

Additional coating substances may also be used, for example, non lipid-based materials (see for example, U.S. Pat. Nos. 6,723,358; and 4,230,687, which are incorporated herein by reference), for example sugars or other carbohydrate-based components that are water soluble. The bacteriophage, or phage component, or both, in the composition of the present invention may also be coated with other substances that are not food grade. Other additive molecules may be added to the coating substance; such additives may include antioxidants, sugars, proteins or synthetic materials.

The process of lipid-based encapsulation protects the bacteriophages, phage components, or both, to some extent from a harsh environment the bacteriophages and/or components may be exposed to, for example, the low pH environment over a range of fermenting liquid feed conditions, or the digestive system of an animal. The lipid-based material selected for encapsulation should also exhibit the property that it breaks down within a desired environment so that the bacteriophages and/or phage components are released. For example, digestive enzymes may degrade the encapsulating material and assist in the release of the bacteriophages and/or phage components within the gut of an animal, or enzymes within the fermenting liquid feed may assist in the release of some of the bacteriophages and/or phage components from encapsulation. As a result, several materials for encapsulating the bacteriophages and/or phage components may be used so that if desired, there is selected release within the fermenting liquid feed, and release within an animal's gut, while at the same time protecting the bacteriophages, phage components, or both. In addition, bacteriophage and/or phage components that are encapsulated using non lipid-based materials will dissolve in water, releasing bacteriophages or phage components immediately, or soon after mixing with the liquid feed medium. The bacteriophage and/or phage components may also be released in a time-controlled fashion depending upon the formulation selected, or whether the preparations are provided within a capsule or tablet form. The capsule or tablet formulations may assist in the timed release of the bacteriophage and/or phage components within the liquid feed medium. Therefore, mixtures of controlled release bacteriophages, phage components, or both that are admixed or encapsulated with different materials may be combined and mixed with animal feed, liquid animal feed, or otherwise administered to an animal.

The immobilized, lyophilized, and/or encapsulated bacteriophages and/or phage components, may also be provided in a capsule form. By “capsule form”, it is meant that the immobilized, lyophilized, and/or encapsulated phages or phage components, or both, are provided in a capsule for example a soft capsule, that may be solubilized within an aqueous environment. The capsule may be made of any suitable substance known in the art, for example, but not limited to gelatin, shellac, wax, synthetic or other compounds.

The immobilized, lyophilized, and/or encapsulated bacteriophages and/or phage components, may also be provided in a tablet form. By “tablet form”, it is meant that the immobilized, lyophilized, and/or encapsulated phages, or phage components, or both are provided in a pressed tablet that dissolves in an aqueous environment. The tablet may be made of any suitable substance known in the art, by any suitable method known in the art. For example, the tablet may comprise binders and other components necessary in the production of a tablet as are known to one of skill in the art. The tablet may be an immediate release tablet, where the bacteriophages and/or phage components are released into the liquid feed upon dissolution of the tablet, or may comprise a timed-release composition, where the bacteriophages and/or phage components are released within an aqueous environment, including the liquid feed, animal gut, or both in a time-dependent manner. See WO 02/45695; U.S. Pat. No. 4,601,894; U.S. Pat. No. 4,687,757, U.S. Pat. No. 4,680,323, U.S. Pat. No. 4,994,276, U.S. Pat. No. 3,538,214, US (which are incorporated herein by reference) for several examples of time-release formulations that may be used to assist in the time controlled release of bacteriophage, or phage components within aqueous environments.

The antibacterial composition of the present invention, in a liquid form, a dry form, including bacteriophages and/or phage components that are lyophilized or adsorbed onto a matrix, encapsulated, or within a capsule or tablet form, or a combination thereof, may be mixed with an animal feed, or a liquid animal feed to produce a treated animal feed, or a treated liquid animal feed, and helps reduce the amount of bacteria in the feed. This treated feed, in either liquid or solid form, may be used to feed any livestock, including swine, or poultry. If controlled release bacteriophages and/or phage components, are used either alone or in combination with un-encapsulated bacteriophages and/or phage components to treat the feed, then in addition to reducing the bacteria content of the feed, further reduction in the contamination of the animals with bacteria, or within animal waste may also be obtained. The use of controlled release bacteriophages, phage components, or a combination thereof, aids in ridding the animal of bacteria already present in the gut prior to further processing of the animal.

By the term “animal feed”, it is meant an animal feed that is dry, or that comprises less than about 25% w/w moisture content. Preferably the moisture content is from about 5 to about 20% w/w, or from about 10 to about 15% w/w. Animal feed may generally comprise a cereal component that may include wheat, barley, soybean, wheat bran and other cereals, and a non-cereal component that may include vitamins, minerals, protein, fat, and other supplements. However, other components may also be present in the animal feed. The definition of the composition of animal feed is not meant to be limiting in any manner.

The animal feed may also be a liquid animal feed. By the term “liquid animal feed”, it is meant an animal feed that is a mixture of water and feed and includes a non-fermented liquid feed (NFLF), and a fermented liquid feed (FLF). Liquid animal feed generally has a cereal component that may comprise wheat, barley, soybean, wheat bran and other cereals, and a non-cereal component that may comprise vitamins, minerals, protein, fat, and other supplements. There are two types of liquid feed: non-fermented and fermented. Non-fermented liquid feed is a mixture of feed and water made immediately before feeding. Fermented liquid feed is a mixture of feed and water that is stored at a given temperature, for a given amount of time, to allow fermentation to begin prior to feeding to the animals. Fermentation of the complete feed, or of only the cereal component can be done. Natural fermentation of the feed, initiated by the natural flora present in the feed, can produce sufficient lactic acid to have a beneficial effect. Alternatively, lactic acid bacteria (LAB) may be added to inoculate the liquid feed (as described by Mann in U.S. Pat. Nos. 6,326,037; 6,699,514; and published U.S. Patent Application 2001/0055633, which is incorporated herein by reference). Any type of liquid feed, for example, NFLF, FLF, FLF comprising LAB, can be utilized in the method of the present invention. Furthermore, other components may also be present in the liquid animal feed. The definition of the composition of liquid animal feed is not meant to be limiting in any manner.

Fermentation of the liquid feed may be accomplished by any method known in the art. For example, which is not to be considered limiting in any manner, the liquid feed may be prepared by mixing meal and water in a ratio from about 1.5:1 to about 4:1, or any amount there between, and storing in a closed tank under agitation at a temperature in the range of about 15° C. to about 30° C. for a time from about 24 hrs to about 10 days, or any amount there between. In an alternate non-limiting example taught by Canibe and Jensen ((2003) J. Anim. Sci., 81, 2019-2031; which is incorporated herein by reference), meal is mixed in water in a 1:2.5, and is stored at 20° C. in a closed tank under agitation for a period of 4 days.

A treated animal feed is an animal feed admixed with an effective amount of an antibacterial composition having one or more than one strain of bacteriophage, one or more phage components from one or more than one strain of bacteriophage, or a combination thereof. The animal feed may be mixed with either a dry or a liquid form of the antibacterial composition. The treated animal feed, or treated liquid animal feed, comprises an effective amount of an antibacterial composition. The treated animal feed may be prepared by any method known in the art. For example, the antibacterial composition may be admixed with the animal feed in a dry form, for example but not limited to, a powder, or a lyophilized preparation may be admixed with the animal feed, or the antibacterial composition may be applied to the animal feed in a liquid form, for example, as a spray, drench, or drip, to produce a treated animal feed. The treated animal feed may then be dried. The effective amount of antibacterial composition having one or more than one strain of bacteriophage, one or more phage components from one or more than one strain of bacteriophage, or a combination thereof, is from about 10³ pfu/g to about 10¹³ pfu/g dry wt of animal feed; for example, from about 10⁵ pfu/g to about 10⁹ pfu/g dry wt of animal feed. In a further example, the amount of the one or more than one strain of bacteriophage, one or more phage components from one or more than one strain of bacteriophage, or a combination thereof, may be from about 10⁶ pfu/g to about 10⁸ pfu/g dry wt of animal feed.

The liquid animal feed may be fermented (FLF) or non-fernented liquid feed (NFLF). When NFLF is used, the bacteriophages and/or phage components may be added to the feed prior to mixing, or after mixing with water. When using FLF, the bacteriophages or phage components can be added prior to, during, or after fermentation. For example, the bacteriophages or phage components, or both, can be added prior to fermentation of the feed. In a more specific example, the bacteriophages and/or phage components can be added prior to fermentation. An amount of from about 10³ pfu/ml to about 10¹¹³ pfu/ml liquid feed may be used, or any amount therebetween; for example, 10³, 10⁴, 10⁵, 20⁶, 10⁷, 10₈, 10⁹, 10¹⁰, 10¹¹, 10¹², 10¹³ pfu bacteriophages and/or phage components can be added per ml liquid feed. In a non-limiting example, from about 10⁵ pfu/ml to 10⁹ pfu/ml of liquid animal feed. In a further example, the amount of the bacteriophages, phage components, or combination thereof, may be from about 10⁶ pfu/ml to about 10⁸ pfu/ml of liquid animal feed. As the bacteriophages are typically active in an environment comprising a pH of greater than 2.5, bacteriophages may be added to FLF (at a pH typically in the pH range of about 3 to about 9, or any amount therebetween, for example a pH of about 5) and still exhibit biological activity. If a lower pH is observed within the FLF, for example if lactobacteria are introduced to the liquid feed, then bacteriophages, encapsulated bacteriophages, or a combination thereof, may be used to treat the FLF with bacteriophages being added prior to addition of the lactobacteria. When the bacteriophages or phage components are added prior to fermentation, the addition can be before or after mixing of the feed with water.

The present invention can be used for animal feed or liquid animal feed destined for any type of animal, including but not limited to animals for agricultural use. For example, but without wishing to be limiting in any manner, the treated animal feed, or treated liquid animal feed, made according to the present invention, may be used for feeding animals for agricultural use, including but not limited to poultry, such as chicken or turkey, and the like; swine; livestock such as horses, goats, sheep, beef cattle, dairy cattle and bison, and the like; domesticated animals, such as household pets including cats, dogs, and the like; fish and shellfish; as well as animals within petting zoos or other animal holding systems. However, it is to be understood that the controlled release bacteriophage, phage components, or both may be administered to an animal via other routes including but not limited to orally, imbibation, inhalation, injection, intramuscular, intraperitoneal, intrathecal, vaginal, rectal, topical or a combination thereof, as required.

The animal should receive the one, or more than one controlled release bacteriophages or phage components in any amount effective for reducing the population of target pathogen in the animal. For example, the controlled release bacteriophages can be administered at a dosage in the range of about 10⁵ to about 10¹³ pfu per animal per day, or any amount therebetween, for example, about 10⁵, 10⁶, 10⁷, 10⁸, 10⁹, 10¹⁰, 10¹¹, 10¹² or 10¹³ pfu per animal day for the desired period of time. For example, and without wishing to be limiting, the bacteriophages may be administered in a treatment dosage of about 10⁹ to about 10¹³ pfu per day, or about 10⁸ to about 10²² pfu per day, or about 10⁹ to about 10¹⁰ pfu per day. The treatment period may be for a period of 1 to about 12 days, or any amount therebetween, for example 1, 2, 3, 4, 5, 6, 7, 8, 9, 10, 11, or 12 days prior to further processing of the animal. Alternatively, a maintenance dosage of about 10⁵ to about 10¹⁰ pfu per day may be used, or any amount therebetween. For example, the maintenance dose may be about 10⁵, 10⁶, 10⁷, 10⁸ or 10⁹, or 10¹⁰ pfu per day for a desired period of time. For example, and without wishing to be limiting, the desired maintenance period may be from about 10 to about 180 days, or any amount therebetween; for example, the maintenance period may be 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 days, or any amount therebetween. In a non-limiting example, the maintenance period may be about 30 to about 90 days, or about 30 to about 60 days. Alternatively, the administration of controlled release bacteriophages or phage components may be done in a treatment dosage of about 10⁹ to about 10¹³ per day, or any amount therebetween, for a period of 1 to about 12 days, or any amount therebetween, for example 1, 2, 3, 4, 5, 6, 7, 8, 9, 10, 11, or 12 days, followed by a maintenance dosage of about 10⁵ to about 10¹⁰ pfu, or any amount therebetween, for a desired period of time, for example but not limited to about 10 to about 180 days, about 30 to about 90 days, or about 30 to about 60 days, or any amount therebetween, prior to further processing of the animal.

Therefore, the present invention provides a method for reducing a population of one or more than one target pathogen in an animal comprising, administering one or more than one controlled release bacteriophage strain or phage component, or both, to the animal, such that the one, or more than one bacteriophage strain is released in vivo and adsorbs to the one, or more than one target pathogen, thereby reducing the one, or more than one pathogen from the animal. Furthermore, in the step of administration, the one or more than one controlled release bacteriophage strain or phage component, or both may for example, be administered to the animal for a period of about 1 to about 12 days, or from about 5 to about 7 days, or any amount therebetween, after which time the animal may be sent off, for example for slaughter. In this case, as a result of the treatment period, the pathogen load in the animals, for example but not limited to, E. coli O157 is reduced or eliminated for an additional 48-72 hrs during which time the animal will be slaughtered. This method ensures that animals going to slaughter comprise a reduced pathogen load and that cleaner animals are being processed within the processing plant.

The present invention also provides a method for reducing a population of one, or more than one target pathogen present in an animal, comprising, administering one or more than one controlled release bacteriophage strain or phage component, or both, to the animal at a dosage from about 10⁵ to about 10¹³ pfu per animal per day for a desired period of time, such that the one or more than one controlled release bacteriophage strain, or phage components, is released in vivo and acts to clear the one or more than one pathogen from the animal.

By administering controlled release bacteriophage or phage components to an animal using a treatment, maintenance, or both regimes as described above, the target bacterial populations amounts decrease within the animal, the animal waste, and within the holding or rearing enclosure, such as a feedlot, in general. Using the method of the present invention reduction of target pathogens may be achieved thereby increasing safety of the food supply for consumption by humans.

Therefore, the present invention also provides a method for reducing a population of one, or more than one target pathogen present within an animal holding system, for example but not limited to a holding enclosure, such as a feedlot, a rearing enclosure, for example a barn or pen, a petting zoo and the like. The method comprising, administering one or more than one controlled release bacteriophage strain, or phage components that are capable of adsorbing to and killing the target pathogen, to animal feed, drinking water, an animal, or a combination thereof, such that the one or more than one controlled release bacteriophage strain or phage component, or both, is released within the feed, the drinking water, a digestive tract of the animal, animal waste, or a combination thereof, and reduces the population of the one or more than one target pathogen within the animal holding system.

The present invention further provides treatment protocols for the reduction of pathogens, for example but not limited to E. coli, Salmonella, Campylobacter, and Staphylococcus, in animals. Without wishing to be limiting, animals to be shipped, or going to slaughter, may be treated with one or more than one controlled release bacteriophage strain, phage component, or both, 5-7 days before being shipped. Using this approach, the pathogen level of the animals will be reduced to low levels by day 3-5 of the treatment, thus allowing safe shipment of the animals from approximately day 4 of treatment onwards. This provides a “safe shipping and processing” window during which the animals from the holding system, for example but not limited to a holding or rearing enclosure, such as a feedlot, can be shipped and processed safely.

In addition, the present invention provides a use of one or more than one bacteriophage strain or phage components, or both, for delivery to animal manure to prevent the spread of bacterial infections through the manure. The one or more than one bacteriophage and/or phage components may be delivered directly to the manure (ex vivo) in an un-encapsulated form, or may be administered to the animal in an encapsulated or controlled release form for delivery to the manure through the animal's gut.

The presence of bacteriophages against a target pathogen in the manure may be beneficial in preventing the spread of bacterial infections caused by various pathogens. In addition to reducing the spread of E. coli O157:H7, which can cause serious health issues in humans, bacteriophages can also reduce the counts of Staphylococcus, which can infect the teats and udder of cattle and cause mastitis. In addition, Treponema infections, causing hoof disease, may be treated in this manner by acting as a foot bath when the animals are walking in the pen.

The present invention will be further illustrated in the following examples.

### EXAMPLES

### Example 1

### Isolation, Amplification and Titration of Phage

Bacteriophages were isolated from manure samples obtained from dairy and beef farms across Canada. Manure samples were allowed to react with E. coli O157:H7 and plated onto agar plates. Any phage plaques obtained were isolated and purified as per standard phage purification protocols (Maniatis et al (1981) Molecular cloning: a laboratory manual, Cold Spring Harbor Laboratory, Cold Spring Harbor, N.Y.).

Purified phages isolated as outlined above were amplified using the isolation strain of E. coli O157:H7. Purified phage and bacteria were mixed together, let stand at room temperature for 10 minutes, and amplified according to standard protocols commonly used in the art (Maniatis et al (1981) Molecular cloning: a laboratory manual, Cold Spring Harbor Laboratory, Cold Spring Harbor, N.Y.). Amplified samples in LB broth were filter sterilized and used.

Concentrations of bacteriophage solutions were determined using standard phage titration protocols (Maniatis et al (1981) Molecular cloning: a laboratory manual, Cold Spring Harbor Laboratory, Cold Spring Harbor, N.Y.). Preparations containing phages were diluted with LB, mixed and incubated with E. coli O157:H7 for 10 minutes and plated onto agar plates. The concentration of phages was determined from the number of plaques obtained at the different dilutions and multiplying with the appropriate dilution factor.

### Example 2

### Immobilization of Phages

E. coli O157:H7 specific phages P10 and R4, prepared as described in example 1, were immobilized on two different matrices: powdered milk (fat free) and soya protein. Both milk powder (Carnation) and soya protein (Supro) were obtained off-the-shelf from local food stores. Identical protocols were used for both materials.

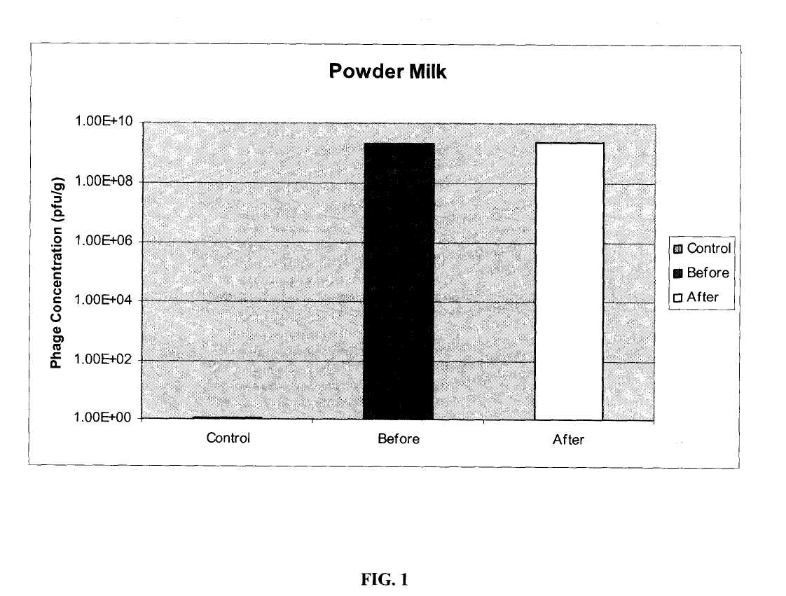

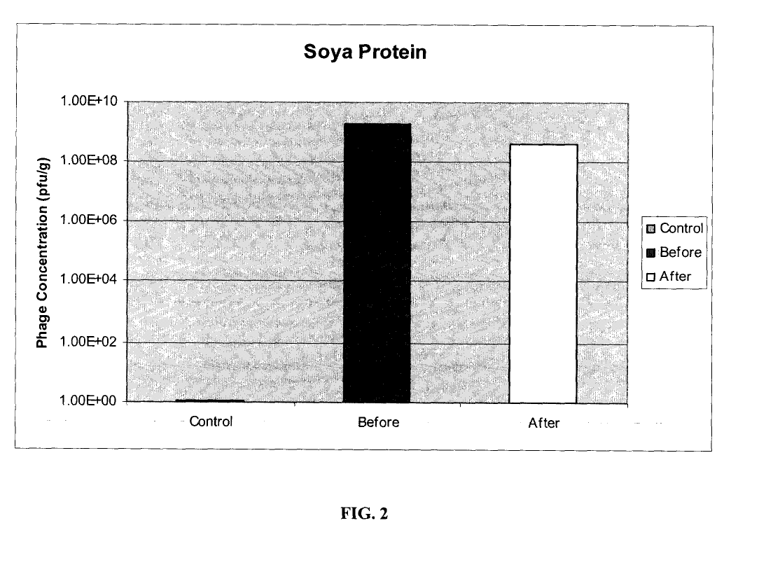

50 g of powder (powdered milk or soya protein) was spread in a glass dish. Phages in solution were uniformly sprayed onto each powdered matrix. Varying titers of phages, ranging from 10⁵ pfu/g to 10⁹ pfu/g, were used with powdered milk, each yielding similar results. The phage-powder was mixed and dried at 37° C. for 2 hours, or until completely dried. The resulting bacteriophage composition was ground into a fine powder, with particle sizes in the range of 50-600 μm and an average particle size of 200 aim. 0.5 grams of each powdered bacteriophage composition was re-suspended in 10 ml of reverse-osmosis (RO) water and the recovery of phages tested. Powdered milk or powdered soya protein in the absence of bacteriophages was used as a control. The results for bacteriophage compositions prepared using dry milk power as the matrix are presented in FIG. 1. Results for bacteriophage compositions prepared using soy protein as the matrix are presented in FIG. 2.

For phage immobilized on powdered milk, the results show that phage can be recovered from the bacteriophage composition and no loss in activity is observed. FIG. 1 shows that the phage titer obtained after immobilization (“After”) is similar to the amount of phage added to the powder (“Before”). Similar results are observed for bacteriophage compositions comprising soy protein (FIG. 2; “After”: immobilized phage; “Before” amount of phage added to matrix).

These results also show that immobilized phages are readily released from a matrix when introduced to an aqueous medium. The results shown in FIGS. 1 and 2 are for phage directed to E. coli, the same results are obtained with bacteriophage directed to Salmonella and Campylobacter.

### Example 3

### Encapsulation of Bacteriophage Compositions

Bacteriophage compositions were prepared as described in Example 2, and encapsulated generally as described in US publication 2003/0109025 (which is incorporated herein by reference), with some modifications to preserve the activity of the phages. Briefly, 400 g of immobilized phage and 1.2 kg of vegetable fatty acids were used for encapsulation. The maximum temperature attained by the encapsulated phage preparation was 39° C. Bacteriophage compositions are also prepared as a tablet using standard methods for example as described in WO 02/45695; U.S. Pat. No. 4,601,894; U.S. Pat. No. 4,687,757, U.S. Pat. No. 4,680,323, U.S. Pat. No. 4,994,276, U.S. Pat. No. 3,538,214, where the pharmaceutical agent is replaced with immobilized phage as prepared in Example 2.

Once the coating operation was complete, the encapsulated immobilized phage particles were collected and stored in airtight containers. The average particle size was between 100 and 1000 μm. Tablets comprising bacteriophage are also stored in airtight containers.

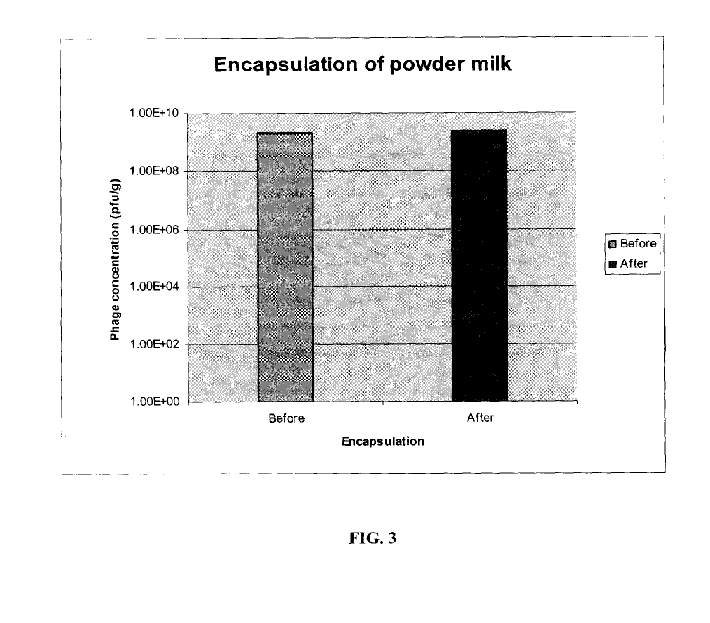

The effect of encapsulation on the titer of bacteriophage compositions immobilized on milk powder was determined by determining the activity of the immobilized phage preparation before (“Before”, FIG. 3) and after (“After”, FIG. 3) encapsulation. For this analysis, encapsulated bacteriophages were re-suspended, and ground using a blender. The re-suspended encapsulated bacteriophages were blended in order to disrupt the encapsulated particles and release the bacteriophages. 0.5 g of encapsulated immobilized phage was mixed with 45.5 ml of re-suspension media (LB Broth or RO Water), and 250 μl of antifoam agent was added to prevent foaming upon grinding. The results of this analysis are shown in FIG. 3.

Similar results are obtained using tableted bacteriophage.

These results demonstrate that bacteriophages can be recovered from an encapsulated, or tableted, bacteriophage composition, and encapsulation or tableting does not inactivate the immobilized phage. The results shown in FIG. 3 are for phage directed to E. coli; similar results are obtained with bacteriophages directed to Salmonella and Campylobacter.

### Example 4

### Stability and Release of Encapsulated Bacteriophages

Phages were immobilized, encapsulated and tableted as described in Example 3. The release of encapsulated immobilized phages upon physical or chemical disruption was tested in the following manner: 0.5 g of encapsulated immobilized phage was mixed with 45.5 ml of re-suspension media (LB Broth or RO Water). 250 μl of antifoam agent was used to prevent foaming upon grinding. A control sample of encapsulated immobilized phages was prepared as described above, but not subjected to grinding, to determine the non-specific leaching of encapsulated bacteriophages within the re-suspension medium. Tableted bacteriophages are processed in a similar manner.

The stability of the encapsulated bacteriophages at low pH was also examined. After re-suspension (as outlined above), the encapsulated immobilized phages were incubated for 30 or 60 min at pH 2.15, neutralized to pH 7.0 using NaOH, then ground using a blender; another sample (control) was resuspended and immediately ground. Both the control and test samples were filter sterilized using a 0.45 μm syringe filter prior to use. Tableted bacteriophages are processed in a similar manner.

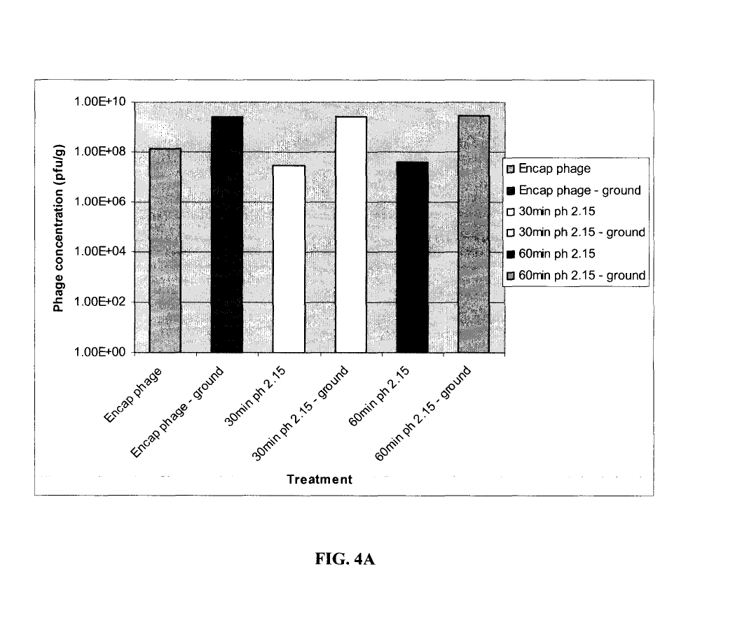

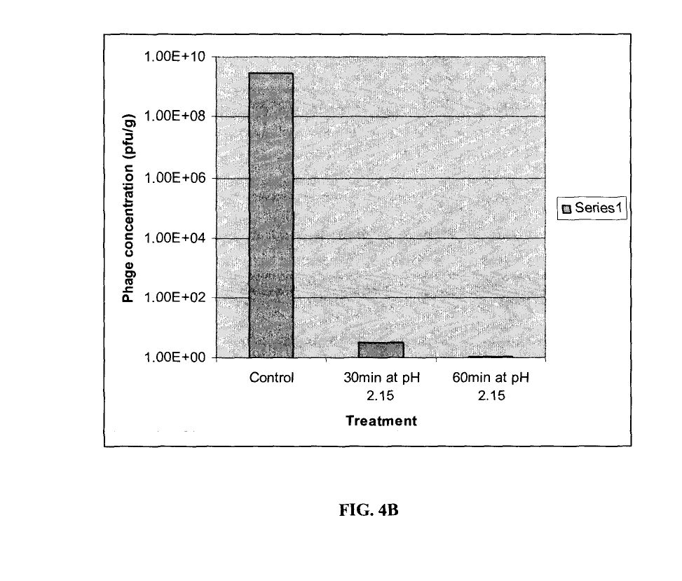

FIG. 4A shows the results of these analyses. The data show that resuspension of the encapsulated immobilized phage results in phage concentrations of about 1×10⁷ pfu/g. Similarly, incubation of the phages at pH 2.15 alone does not cause significant release of phages (phage concentration of about 1×10⁶ pfu/g after 30 minutes, or a phage concentration of about 3×10⁷ pfu/g after 60 minutes). However, following grinding and disruption of the encapsulated bacteriophage particles, the amount of phage released is about the same amount as was loaded onto the milk powder for immobilization (about 5×10⁹ pfu/g). Incubation of non encapsulated and non immobilized phages at pH 2.15 for 30 and 60 minutes however resulted in essentially complete loss of phage infectivity (FIG. 4B). Similar results are obtained using tableted bacteriophages.

These results demonstrate that bacteriophages may be released following disruption of encapsulated bacteriophage particles. Furthermore, these results shows that encapsulated bacteriophages may be exposed to a pH of 2.15 for prolonged period of time, with little or no loss in activity (titer). The results for non-encapsulated and non-immobilized bacteriophages are consistent with the results of Jepson and March (2004, Vaccine, 22:2413-2419), where a dramatic loss of viability of bacteriophages was observed after only 5 minutes at pH below pH 2.2. This loss in activity is obviated by encapsulation of the bacteriophages as described in the present invention. Similar results are obtained using tableted bacteriophages.

The results shown in FIGS. 4A and 4B are for phage directed to E. coli; similar results are obtained with bacteriophages directed to Salmonella and Campylobacter.

### Example 5

### Stability of Immobilized Phage

Bacteriophages were immobilized on a matrix, in this case milk powder as described in Example 2 and the material was stored at either room temperature (RT) or at 4° C. (4C) in airtight containers. Samples were obtained at different time points, and phage titers determined, over a period of 10 months. The initial phage concentration was 3×10⁶ pfu/g.

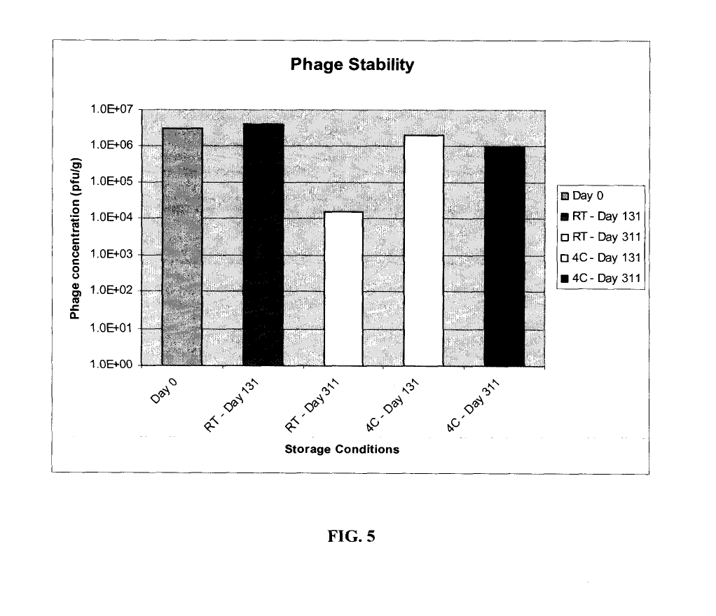

FIG. 5 shows that the immobilized phages (bacteriophage composition) are stable at either room temperature or 4° C. for at least 131 days (4.5 months), and are stable for at least 311 days (10 months) at 4° C. Addition of a desiccant, or storage of the bacteriophages in a desiccated environment may further increase the viability of the bacteriophage composition.

### Example 6

### Treatment of Cattle Naturally Contaminated with E. Coli O157:H7, Using Encapsulated Bacteriophages

In this dose ranging study, auction market-derived, exotic crossbred feedlot steer calves were used in all phases. The individual animal weight of study animals was between 200 kg and 250 kg (441 lbs and 551 lbs). On Day −4, 50 to 100 gram fecal samples were obtained from each animal and 40 animals culture-positive for E. coli O157:H7 were identified for use in the study.

The animals selected for this study were allocated to different groups using a computer generated randomization table to one of three experimental groups: a control group or a phage-treated group (10¹⁰ pfu/animal/day). There were 20 animals in each group, with 10 animals being housed each pen.

The feed was formulated to meet or exceed the nutritional requirements of feedlot animals, and was tested to ensure that it was free of E. coli O157:H7 and E. coli O157:H7 bacteriophages prior to the study. The animals were allowed to consume feed and water on an ad libitum basis throughout the feeding period.

For animals in the control group, 10 g per animal of control encapsulation material alone (not containing phage) was thoroughly mixed into their 1 day's ration. The encapsulation material was applied to the feed for a 5 day period.

For animals in the phage-treated groups, a cocktail of 3 bacteriophages immobilized (see Example 2) and encapsulated (see Example 3) were used. 10 g per animal of the encapsulated bacteriophage preparation (equivalent to 10¹⁰ pfu/animal/day) at the appropriate dose was thoroughly mixed with 1 day's ration of feed, such that the phages were delivered to the animals over a 24 hr period. The encapsulation material was applied to the feed for a 5 day period.

The animals were housed in an enclosed a level 1 facility.

Duplicate 50 to 100 gram fecal samples were collected on Days 0, 1, 2, 3, 4, 5, 6, 7, 8, 9, and 10 of the study. One sample was used to determine bacteriophage counts, and the other was used to determine E. coli O157:H7 shedding and total coliform counts. A new palpation sleeve was used for each animal to collect the fecal samples, which were then transferred to a suitable container and stored at 4° C. prior to shipment.

In addition to blood, 10 to 20 grams of the following specific tissues were sampled upon sacrifice: liver, spleen, kidney, triceps, semitendinosus and diaphragm.

The E. coli O157:H7 shedding, total E. coli and coliform fecal count and total bacteriophage count were compared between the experimental groups using appropriate descriptive and analytical statistical methods, controlling for the fact that the measurements represent repeated measures within each animal.

### Reduction in E. Coli O157:H7 Counts in Cattle Naturally Carrying the Pathogen

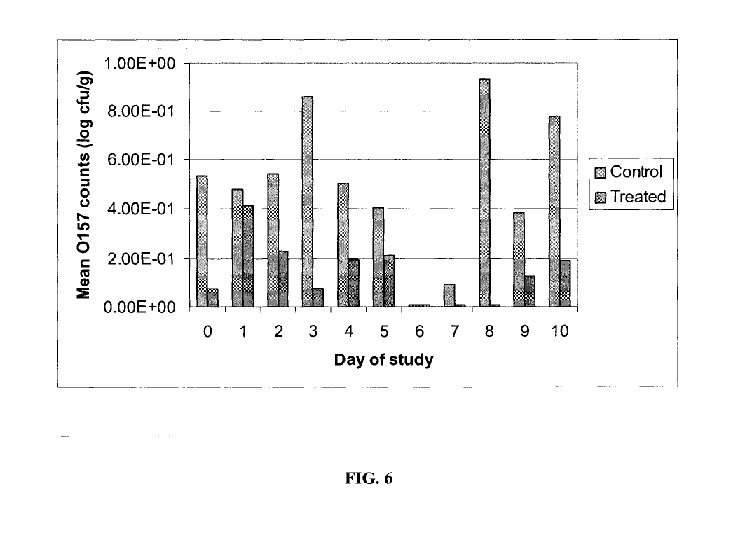

Treatment of the test groups of calves with phages administered from Day 2 to Day 4 resulted in a significant decrease in E. coli O157:H7 shedding as compared to the control group (see FIG. 6). The bacterial load was consistently higher in the control group as compared to the phage-treated group. The bacterial shedding remained higher in the control group compared to the phage-treated group, even after the last day of treatment, suggesting recontamination due to higher pathogen load in the pen.

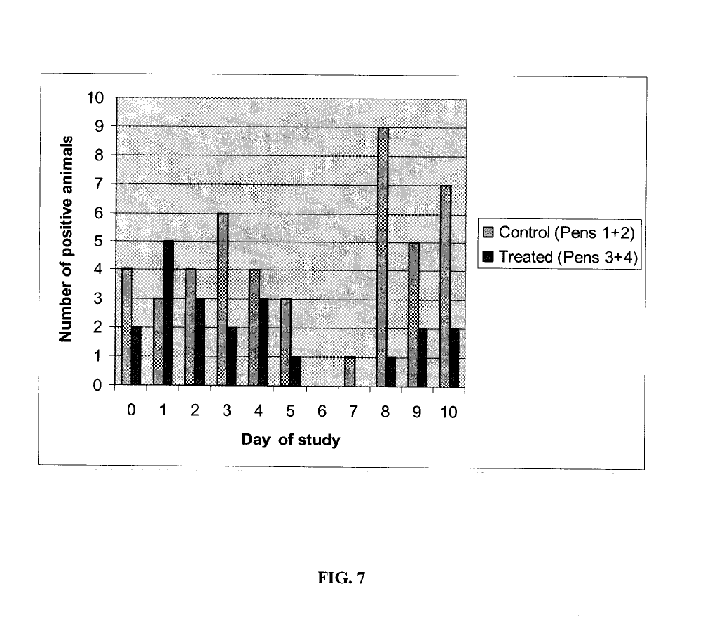

In addition to the level of E. coli O157:H7 shedding, the total number of animals shedding in each of the treatment period (Day 1-6) as well as the post-treatment period (Days 7-10) was lower in the phage-treated groups compared to the control group (see FIG. 7). This demonstrates the effectiveness of encapsulated phages in reducing pathogen load in farm animals as well as reducing the number of animals carrying the pathogen.

Similar results were obtained in studies using bacteriophage dosages of 10⁹ pfu/animal/day and 10¹¹ pfu/animal/day.

Shedding of E. coli O157:H7 Phases from the Animals

The shedding of the three phages used in the above treatment protocol was followed over the course of the study.

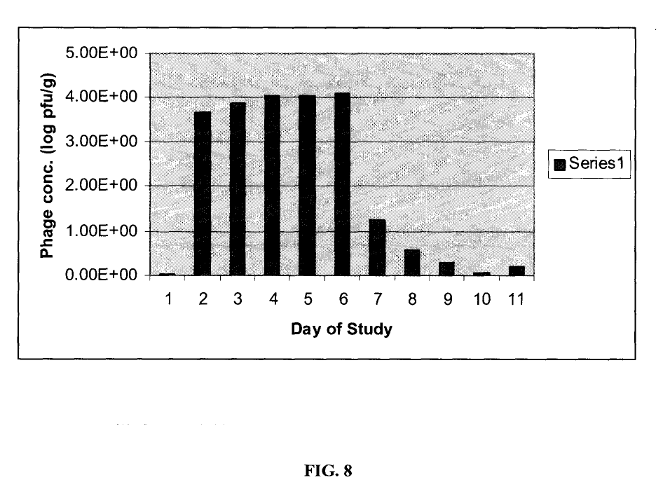

An increase in the number of E. coli O157:H7-specific shed in the phage-treated group was observed after administration, reaching high numbers on day 1 and remaining there for the next 4 days (see FIG. 8). Phage shedding levels reached below 100 pfu/g, by day 6, which is well within the background level of phage shedding observed in many feedlot animals.

The data suggest that phages administered to calves are delivered efficiently to the intestines, past the acidity of the abomassum and maintained at the desired levels over the period of phage administration. However, phages are eliminated from the system in about 1-2 days after the last phage dose, suggesting that there is no retention of phages in the animals due to non-specific binding. In the course of screening feedlot animals, low levels of native phage in many animals were observed, therefore it may not be necessary to bring the phage count below background levels at the end of the treatment.

### RFLP Analysis of Phages

All phages recovered in the course of the study were analyzed by RFLP to determine if the phages isolated from the phage-treated animals were the same phages as those used for the treatment. Phage DNA was purified from the plaques and used for analysis. Well-isolated plaques were used for amplification and DNA prepared from the lysates.

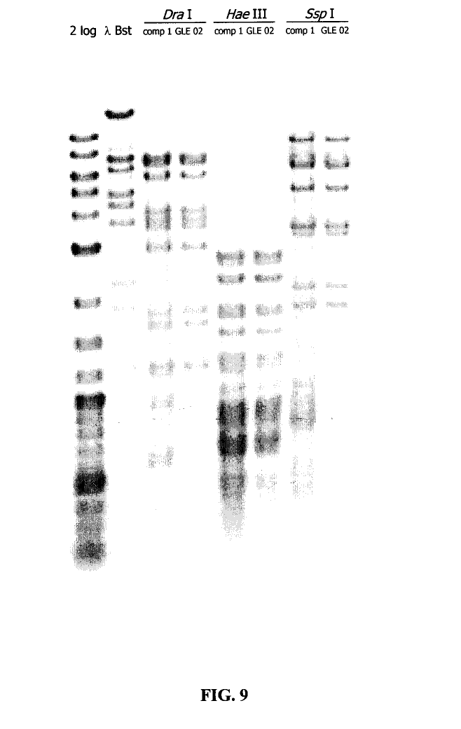

RFLP patterns obtained from phage DNA isolated from fecal samples indicated that all three phages used for the treatment survived passage through the abomasums and reached the small intestine. The RFLP pattern of the individual phages also revealed that there was no change to the administered phages at the genomic level as a result of passing through the GI tract in calves. A representative RFLP pattern of one of the administered phages using three different enzymes is provided in FIG. 9.

These data further point to the usefulness of these phages as effective anti-infective agents.

### Tissue Uptake of Phages

Animals were sacrificed at the end of the study (Day 10) and tissues harvested at necropsy. The following tissues were harvested: liver, spleen, kidney, triceps, semitendinosus and diaphragm. The tissue samples were homogenized and tested for phages. No phages were detected in any of the tissues analyzed, nor in the blood.

Similar results were obtained in studies using bacteriophage dosages of 10⁹ pfu/animal/day and 10¹¹ pfu/animal/day.

### Treatment Protocols

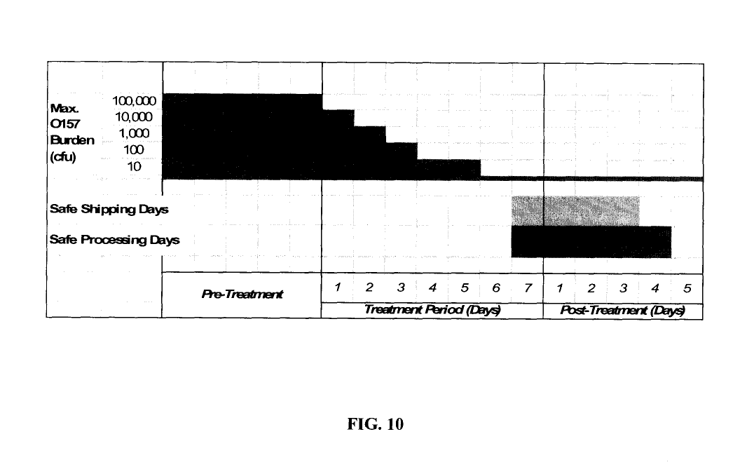

The proposed treatment protocol for the reduction of E. coli O157:H7 in cattle prior to slaughter is as follows: all animals going to slaughter are treated with a phage cocktail one week before being shipped. Using this approach, the pathogen level of all the animals will be reduced to very low levels (close to or below the level of detection as seen in the present studies) by day 3-5 of the treatment thus allowing safe shipment of the animals from day 4 of treatment onwards. This provides a one week “safe shipping and processing” window during which the cattle from the feedlot can be shipped and processed safely (see FIG. 10). This also gives the farmer a ±3 day window around the shipping date.

All citations are hereby incorporated by reference.

The present invention has been described with regard to one or more embodiments. However, it will be apparent to persons skilled in the art that a number of variations and modifications can be made without departing from the scope of the invention as defined in the claims.

1. A method for reducing a population of one or more than one target pathogen in an animal comprising, administering one or more than one controlled release bacteriophage strain or phage tail, or both, to the animal, such that the one, or more than one bacteriophage strain is released in vivo and adsorbs to the one, or more than one target pathogen, thereby reducing the one, or more than one pathogen from the animal.

2. The method of claim 1, wherein the one or more than one controlled release bacteriophage strain is administered at a treatment dosage of about 10⁵ to about 10¹³ pfu per animal per day from about 1 to about 12 days.

3. The method of claim 1, wherein the one or more than one controlled release bacteriophage strain is administered at a maintenance dosage of about 10⁵ to about 10¹⁰ pfu per animal per day.

4. The method of claim 1, wherein the one or more than one controlled release bacteriophage strain is administered at a treatment dosage of about 10⁵ to about 10¹³ pfu per animal per day from about 1 to about 12 days, followed by a maintenance dosage of about 10⁵ to about 10¹⁰ pfu per animal per day.

5. The method of claim 1 wherein the step of administrating includes oral, inhalation, injection, intramuscular, intraperitoneal, intrathecal, rectal, topical, or a combination thereof.

6. The method of claim 5, wherein oral administration includes providing the one or more than one controlled release bacteriophage strain or phage tail, or both, within animal feed, drinking water, or a combination thereof.

7. The method of claim 1, wherein the one or more than one controlled release bacteriophage, phage tail, or both, are immobilized on a matrix, lyophilized, immobilized on a matrix and encapsulated, lyophilized and encapsulated, provided in a capsule form, provided in a tablet form, or a combination thereof.

8. The method of claim 7, wherein the matrix is selected from the group consisting of skim milk powder, soya protein, albumin powder, single cell proteins, trehalose, manitol, sugar, sugar alcohol, other water-soluble carbohydrate-based materials, synthetic plant-derived plastic, and a combination thereof.

9. The method of claim 8, wherein the one or more than one controlled release bacteriophage, phage tail, or both, is encapsulated using a material selected from the group consisting of vegetable fatty acids, fatty acid, stearic acid, palmitic acid, an animal wax, a vegetable wax, Carnauba wax, and a wax derivative.

10. The method of claim 8, wherein the one or more than one controlled release bacteriophage, phage tail, or both, is encapsulated using a material selected from the group consisting of sugars and soluble non lipid-based materials.

11. The method of claim 1, wherein a phage protein is administered in addition to the one or more than one bacteriophage, phage tail, or both.

12. A method for reducing a population of one, or more than one target pathogen present within a holding system, comprising, administering one or more than one controlled release bacteriophage strain, or phage tail to animal feed, drinking water, an animal, or a combination thereof, such that the one or more than one controlled release bacteriophage strain, or phage tail, is released within the feed, the drinking water, a digestive tract of the animal, manure, or a combination thereof, and adsorbs to, and kills, the target pathogen, thereby reducing the population of the one or more than one target pathogen within the holding system.

13. The method of claim 12, wherein the one or more than one controlled release bacteriophage strain is administered at a treatment dosage of about 10⁵ to about 10¹³ pfu per animal per day from about 1 to about 12 days.

14. The method of claim 12, wherein the one or more than one controlled release bacteriophage strain, or phage tail, is administered at a maintenance dosage of about 10⁵ to about 10¹⁰ pfu per animal per day.

15. The method of claim 12, wherein the one or more than one controlled release bacteriophage strain is administered at a treatment dosage of about 10⁵ to about 10¹³ pfu per animal per day from about 1 to about 12 days, followed by a maintenance dosage of about 10⁵ to about 10¹⁰ pfu per animal per day.

16. The method of claim 12 wherein the step of administrating includes oral, inhalation, injection, intramuscular, intraperitoneal, intrathecal, rectal, topical, or a combination thereof.

17. The method of claim 16, wherein oral administration includes providing the one or more than one controlled release bacteriophage strain or phage tail, or both, within animal feed, drinking water, or a combination thereof.

18. The method of claim 12, wherein the one or more than one controlled release bacteriophage, phage tail, or both, are immobilized on a matrix, lyophilized, immobilized on a matrix and encapsulated, lyophilized and encapsulated, provided in a capsule form, provided in a tablet form, or a combination thereof.

19. The method of claim 18, wherein the matrix is selected from the group consisting of skim milk powder, soya protein, albumin powder, single cell proteins, trehalose, manitol, sugar, sugar alcohol, other water-soluble carbohydrate-based materials, synthetic plant-derived plastic, and a combination thereof.

20. The method of claim 19, wherein the one or more than one controlled release bacteriophage, phage tail, or both, is encapsulated using a material selected from the group consisting of vegetable fatty acids, fatty acid, stearic acid, palmitic acid, an animal wax, a vegetable wax, Carnauba wax, and a wax derivative.

21. The method of claim 19, wherein the one or more than one controlled release bacteriophage, phage tail, or both, is encapsulated using a material selected from the group consisting of sugars and soluble non lipid-based materials.

22. The method of claim 12, wherein a phage protein is administered in addition to the one or more than one bacteriophage, phage tail, or both.

23. The method of claim 1 wherein, in the step of administration, the one or more than one controlled release bacteriophage strain or phage tail, or both is administered to the animal for a period of 5-7 days.

24. The method of claim 23 wherein, following the step of administration, the animal is slaughtered.

25. A method for preventing the spread of infections in an animal caused by one or more than one target pathogen, the method comprising administering one or more than one bacteriophage strain, phage tail, or both, to the animal, such that the one, or more than one bacteriophage strain, phage tail, or both, is released within the digestive tract of the animal, and attach to and kill the target pathogen, thereby reducing the population of the one or more than one target pathogen within animal waste.

26. The method of claim 25, wherein the target pathogen is E. coli O157:H7, Staphylococcus aureus, Treponema, or another pathogen carried in the gastrointestinal tract, or a combination thereof.

27. The method of claim 25, wherein the one or more than one bacteriophage strain, phage tail, or both, is provided as a controlled release bacteriophage strain, phage tail, or both.

28. The method of claim 26, wherein the one or more than one bacteriophage strain, phage tail, or both, is provided as a controlled release bacteriophage strain, phage tail, or both.
